(19) United States
(12) Patent Application Publication
(10) Pub. No.: US 2021/0247347 A1
(43) Pub. Date: Aug. 12, 2021

(54) ELECTRONIC CONDUCTANCE IN BIOELECTRONIC DEVICES AND SYSTEMS

(71) Applicant: Arizona Board of Regents on behalf of Arizona State University, Scottsdale, AZ (US)

(72) Inventors: Stuart Lindsay, Scottsdale, AZ (US); Joshua Sadar, Scottsdale, AZ (US); Quan Qing, Scottsdale, AZ (US)

(21) Appl. No.: 17/173,569

(22) Filed: Feb. 11, 2021

Related U.S. Application Data
(60) Provisional application No. 62/975,748, filed on Feb. 12, 2020.

(51) Int. Cl. G01N 27/327; G01N 27/02

(52) U.S. Cl. CPC G01N 27/3275; G01N 27/026

(57) ABSTRACT

The present disclosure provides devices, systems, and methods related to protein bioelectronics. In particular, the present disclosure provides bioelectronic devices, systems, and methods that utilize a defined electrical potential to maximize electrical conductance of a protein-of-interest, which can serve as a basis for the fabrication of enhanced bioelectronic devices for the direct measurement of protein activity.

### RELATED APPLICATIONS

This application claims priority to and the benefit of U.S. Provisional Patent Application No. 62/975,748 filed Feb. 12, 2020, which is incorporated herein by reference in its entirety for all purposes.

### GOVERNMENT SUPPORT

This invention was made with government support under Grant Nos. HG006323 and R21 HG010522 awarded by the National Institutes of Health. The government has certain rights in the invention.

### FIELD

The present disclosure provides devices, systems, and methods related to protein bioelectronics. In particular, the present disclosure provides bioelectronic devices, systems, and methods that utilize a defined electrical potential to maximize electrical conductance of a protein-of-interest, which can serve as a basis for the fabrication of enhanced bioelectronic devices for the direct measurement of protein activity.

### BACKGROUND

As proteins perform their various functions, movements are generated that underlie these functions. The ability to develop devices, systems, and methods that measure the electrical characteristics corresponding to the fluctuations generated by an active protein can be a basis for label-free detection and analysis of protein function. For example, monitoring the functional fluctuations of an active enzyme may provide a rapid and simple method of screening candidate drug molecules that affect the enzyme's function. In other cases, the ability to monitor the fluctuations of proteins that process biopolymers (e.g., carbohydrates, polypeptides, nucleic acids, and the like) may reveal new information about their conformational changes and how those changes are linked to function. Additionally, diagnostic and analytical devices can be developed to take advantage of the electrical characteristics produced by active proteins, providing new ways to leverage biomechanical properties for practical use.

Bioelectronics research has mainly focused on redox-active proteins because of their role in biological charge transport. In these proteins, electronic conductance is a maximum when electrons are injected at the known redox potential of the protein. It has been shown recently that many non-redox active proteins are good electronic conductors, though the mechanism of conduction is not yet understood. Additionally, most bioelectronic devices use gold for device fabrication. Gold is the most widely-used metal in molecular electronic devices, partly because it is relatively easy to make high-quality molecular monolayers on gold and partly because it is used to make molecular break-junctions, the most common method for mounting molecules in an electrode junction. However, the general malleability of gold also presents challenges for device fabrication. Accordingly, there exists a need for alternative materials and methods for fabricating bioelectronic devices with enhanced conductance, as well as improved composition and geometry.

### SUMMARY

Embodiments of the present disclosure include a bioelectronic device that includes a first electrode and a second electrode separated by a gap, and a protein attached to the first and second electrodes via a linker. In accordance with these embodiments, the electrical surface potential of the first and second electrodes at zero bias is from about 250 mV to about 400 mV on the normal hydrogen electrode scale.

In some embodiments, conductance of the protein is maximized when the surface potential of the first and second electrodes at zero bias is from about 250 mV to about 400 mV. In some embodiments, the surface potential of the first and second electrodes at zero bias is from about 250 mV to about 350 mV. In some embodiments, the first and second electrodes are comprised of a metal or metals that impart a surface potential from about 250 mV to about 400 mV at zero bias.

In some embodiments, at least one of the first and second electrodes comprises a different metal as that of the other electrode. In some embodiments, at least one of the first and second electrodes comprises gold or an alloy thereof. In some embodiments, both the first and second electrodes comprise gold or an alloy thereof. In some embodiments, the first electrode comprises gold or an alloy thereof and the second electrode comprises a different metal or an alloy thereof. In some embodiments, the second electrode comprises palladium or an alloy thereof. In some embodiments, the second electrode comprises platinum or an alloy thereof.

In some embodiments, the device comprises a reference electrode. In some embodiments, the surface potential of the first and second electrodes is maintained at about 250 mV to about 400 mV due to a bias applied between the reference electrode and at least one of the first or second electrode. In some embodiments, the reference electrode comprises a third electrode immersed in an electrolyte solution and in contact with the first and second electrodes.

In some embodiments, the gap has a width of about 1.0 nm to about 20.0 nm. In some embodiments, the first and second electrodes are separated by a dielectric layer.

In some embodiments, the protein is a non-redox protein. In some embodiments, the protein is selected from the group consisting of a polymerase, a nuclease, a proteasome, a glycopeptidase, a glycosidase, a kinase and an endonuclease.

In some embodiments, the linker is attached to an inactive region of the protein. In some embodiments, the linker comprises a covalent chemical bond. In some embodiments the linker comprises a ligand that specifically binds a region of the protein. In some embodiments, the protein is biotinylated. In some embodiments, the linker comprises thio-streptavidin. In some embodiments, the protein and the first and second electrodes are biotinylated, and wherein the linker comprises a streptavidin molecule comprising at least two biotin binding sites.

Embodiments of the present disclosure also include a system for direct electrical measurement of protein activity. In accordance with these embodiments, the system includes any of the bioelectronic devices described herein, a means for introducing an analyte capable of interacting with the protein, a means for applying a voltage bias between the first and second electrodes that is 100 mV or less, and a means for monitoring fluctuations that occur as the chemical entity interacts with the protein.

Embodiments of the present disclosure also include an array comprising a plurality of any of the bioelectronic devices described herein.

In some embodiments, the array includes a means for introducing an analyte capable of interacting with the protein, a means for applying a voltage bias between the first and second electrodes that is 100 mV or less, and a means for monitoring fluctuations that occur as the chemical entity interacts with the protein.

Embodiments of the present disclosure also include a method for direct electrical measurement of protein activity. In accordance with these embodiments, the method includes introducing an analyte capable of interacting with the protein to any of the bioelectronic devices described herein, applying a voltage bias between the first and second electrodes that is 100 mV or less, and observing fluctuations in current between the first and second electrodes that occur when the analyte interacts with the protein.

In some embodiments, the analyte is a biopolymer selected from the group consisting of a DNA molecule, an RNA molecule, a peptide, a polypeptide, or a glycan.

### BRIEF DESCRIPTION OF THE DRAWINGS

FIGS. 1A-1D: Measuring protein conductance under potential control. (A) Illustrating the surface potentials generated when two metals with different work functions are connected to a reference electrode. The molecule, M, is assumed to sit in the middle of the potential gradient generated by the difference in surface potentials of the two metals. (B) STM measurement of protein conductance illustrating streptavidin protein (green) bound to electrodes by thiolated biotin molecules (red). The substrate is held at a potential Vᵣ with respect to a salt-bridged reference electrode. For conductance measurements, a low (10 mM) KCl concentration is used in the bridge, leading to a 360 mV difference with respect to the NHE. (C) Typical current-voltage (IV) curve for a single streptavidin molecule. Black data points are scanning up, red data points are scanning down. The green line is a linear fit yielding the conductance for this particular contact geometry. (D) Conductance distributions derived from many such IV curves for biotin/streptavidin on Au, Pd and Pt electrodes as marked. The dashed lines indicate the positions of peaks II and III in the distribution for the case of Au electrodes.

FIGS. 2A-2D: Streptavidin conductance depends on potential. (A) Rest potentials are measured using a high-impedance voltmeter (V_{REST}) connected between the electrode and a salt-bridged reference electrode. In this case, the KCl concentration is 3M, corresponding to a 210 mV shift relative to the NHE scale. (B) Change in rest potentials with surface functionalization. Points from UHV are translated to the NHE scale using the work function of the NHE. (C) Conductance peak values for a streptavidin molecule as a function of electrode material (as marked, the first listed material is the STM tip, the second the substrate). Green triangles are for reversed combinations for the tip and substrate materials. (D) Conductance peaks measured as a function of potential (Vᵣ in FIG. 1A) for streptavidin on Pd electrodes. Error bars in FIGS. 1C and 1D are uncertainties in fits to the conductance distributions.

FIGS. 3A-3B: An antibody and a polymerase show similar dependence of conductance on potential. (A) Conductance of an anti-DNP IgE molecule for the electrode combinations shown (blue triangles are for reversed tip/substrate combinations). (B) A similar distribution for a doubly-biotinylated Φ29 polymerase trapped between streptavidin functionalized electrodes. Green triangles are reversed metal combinations. Parameters for the Lorentzian fits are given in Table 2.

FIG. 4: UPS spectra for the three metals after in-Situ hydrogen plasma cleaning. The secondary electron emission cutoff was determined using the linear fit method. The work function is the difference in energy between the photon energy and this secondary electron emission cutoff. The work function is a measure of the difference between the vacuum energy level and the Fermi Energy.

FIG. 5: Conductance distributions for the streptavidin-biotin system (gap=2.5 nm) for the tip (first metal listed)−substrate (second metal listed) combinations.

FIG. 6: Conductance distributions for the streptavidin-biotin system (gap=2.5 nm) for the substrate potentials as listed vs. a 10 mM salt-bridged Ag/AgCl reference with Pd electrodes. These potentials are converted to NHE by adding 380 mV.

FIG. 7: Conductance distributions for the DNP-anti DNP IgG system (gap=4.5 nm) for the three metals shown.

FIG. 8: Conductance distributions for the DNP-anti DNP IgG system (gap=4.5 nm) for the tip (first metal listed)−substrate (second metal listed) combinations.

FIG. 9: Conductance distributions for the biotin-SA-Φ29 system (gap=4.5 nm) for the three metals shown.

FIG. 10: Conductance distributions for the biotin-SA-Φ29 system (gap=4.5 nm) for the tip (first metal listed)−substrate (second metal listed) combinations.

FIG. 11: Reversibility of the conductance distributions over the range of surface potential measured (values shown are vs. the 10 mM salt-bridged Ag/AgCl electrode). Fitting parameters are listed in Table 8.

FIG. 12: Tyrosines (yellow) and tryptophans (red) in streptavidin (1VWA), Φ29 polymerase (2PYJ) and an IgE molecule (4GRG) where the codes are PDB IDs.

FIG. 13: FTIR scans from Pd, Pt and Au surfaces modified with thiolated biotin. The top recording is the bulk (disulfide) powder.

FIG. 14: Representative schematic diagram of a molecular junction in which the edges of the bottom gold electrode are sealed, according to one embodiment of the present disclosure.

FIG. 15: Representative schematic diagram of a molecular junction with an additional dielectric over the edges of the first electrode, according to one embodiment of the present disclosure.

FIG. 16: Representative schematic diagram of a completed molecular junction with additional dielectric between the junction metals at the edge of the first electrode, according to one embodiment of the present disclosure.

FIG. 17: Representative schematic diagram of a molecular junction comprising a protein-of-interest, according to one embodiment of the present disclosure.

FIG. 18: Representative schematic diagram of an array comprising a plurality of bioelectronic devices, according to one embodiment of the present disclosure.

### DETAILED DESCRIPTION

Bioelectronics research has mainly focused on redox-active proteins because of their role in biological charge transport. In these proteins, electronic conductance is a maximum when electrons are injected at the known redox potential of the protein. It has been shown recently that many non-redox active proteins are good electronic conductors, though the mechanism of conduction is not yet understood. Embodiment of the present disclosure demonstrate single-molecule measurements of the conductance of three non-redox active proteins, maintained under potential control in solution, as a function of electron injection energy. All three proteins show a conductance resonance at a potential ˜0.7V removed from the nearest oxidation potential of their constituent amino acids. If this shift reflects a reduction of reorganization energy in the interior of the protein, it may explain the long range conductance observed when carriers are injected into the interior of a protein.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As noted herein, the disclosed embodiments have been presented for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, compositions, systems and apparatuses/devices which may further include any and all elements from any other disclosed methods, compositions, systems, and devices, including any and all elements corresponding to detecting protein activity. In other words, elements from one or another disclosed embodiments may be interchangeable with elements from other disclosed embodiments. Moreover, some further embodiments may be realized by combining one and/or another feature disclosed herein with methods, compositions, systems and devices, and one or more features thereof, disclosed in materials incorporated by reference. In addition, one or more features/elements of disclosed embodiments may be removed and still result in patentable subject matter (and thus, resulting in yet more embodiments of the subject disclosure). Furthermore, some embodiments correspond to methods, compositions, systems, and devices which specifically lack one and/or another element, structure, and/or steps (as applicable), as compared to teachings of the prior art, and therefore represent patentable subject matter and are distinguishable therefrom (i.e. claims directed to such embodiments may contain negative limitations to note the lack of one or more features prior art teachings).

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### 2. BIOELECTRONIC DEVICES AND SYSTEMS

Electronic Conductance. Proteins are generally believed to be insulators, practically, because of the need to sustain high external electric fields, and theoretically, because of strong vibronic coupling that traps carriers. Nonetheless, there is ample evidence of long range electronic transport in proteins, although almost all of these prior studies have focused on proteins that contain redox centers, because of their role in biological charge transport and because a considerable body of evidence suggests that optimal electron-tunneling pathways have evolved in these particular proteins. Motivated by a recent theoretical proposal that suggested unusual electrical properties might be a feature of all functional proteins (and not just proteins involved in electron transfer) the electronic conductance of a series of non-redox active proteins was measured. These proteins were maintained under potential control in solution, in conditions that preclude ion currents. Their conductance was high and showed little decay with distance, so long as charge is injected into the protein interior via ligands or other good chemical contacts. This property has important technological consequences. For example, protein molecular wires are self-assembling and transport charge over longer distances than synthetic molecular wires. This conductance has been shown to depend on the conformation of a protein, so that enzymatic processes, such as DNA synthesis, can be followed dynamically with a direct electrical read-out.

However, the mechanism of long-range charge transport in non-redox active proteins is unknown at present. The role that redox centers play in charge-transfer proteins has been demonstrated by electrochemical gating experiments, in which conductance is measured as a function of the electrochemical potential of the surface to which the protein is bound. In redox proteins, the peak conductance coincides with the known redox potential of the active site. As previously described, solvent reorganization energy contributes significantly to the redox potential, and this depends strongly on the solvating medium. Embodiments of the present disclosure demonstrate the existence of a conductance maximum in three non-redox active proteins. The peak potential is almost the same in all three proteins studied, indicating a common transport mechanism. It occurs at a potential that is about 0.7 V less than the redox potential of aromatic amino acids in solution, suggesting that the effective Marcus reorganization energy is reduced by this amount when these same amino acid residues are enclosed in the interior of a protein.

As described further herein, the observation of a conductance resonance is unexpected in a non-redox active protein, if the redox potentials of amino acid residues are taken as a measure of the energy of molecular states in the protein. The observation of similar resonances in three electrochemically inert proteins strongly suggests that the same mechanism controls conductance in all three proteins, and that the energies of the molecular states responsible for transport are located at approximately +300 mV on the NHE scale. In the simplest model of resonant tunneling via a single electronic level, the dependence of conductance on electronic energy is described by the Breit-Wigner formula:

$$G ∝ \frac{{Γ}^{L} {Γ}^{R}}{{( {E}_{0} - E )}^{2} + {( {Γ}^{L} + {Γ}^{R} )}^{2} / 4} = \frac{{Γ}^{2}}{{( {E}_{0} - E )}^{2} + {Γ}^{2}} \qquad ( 3 )$$

The expression on the right-hand side is simplified by assuming that the coupling to the left electrode Γ^{L} is equal to the coupling to the right electrode, Γ^{R} (=Γ), as should apply to the symmetrically-bonded molecule geometry that gives rise to the higher conductance peaks in the embodiments of the present disclosure. This is the Lorentzian function that has been fitted to yield the parameters listed in Table 2, where the listed full width at half maximum is equal to twice the value of Γ in equation 3. The R² values suggest that this choice of fitting function is reasonable.

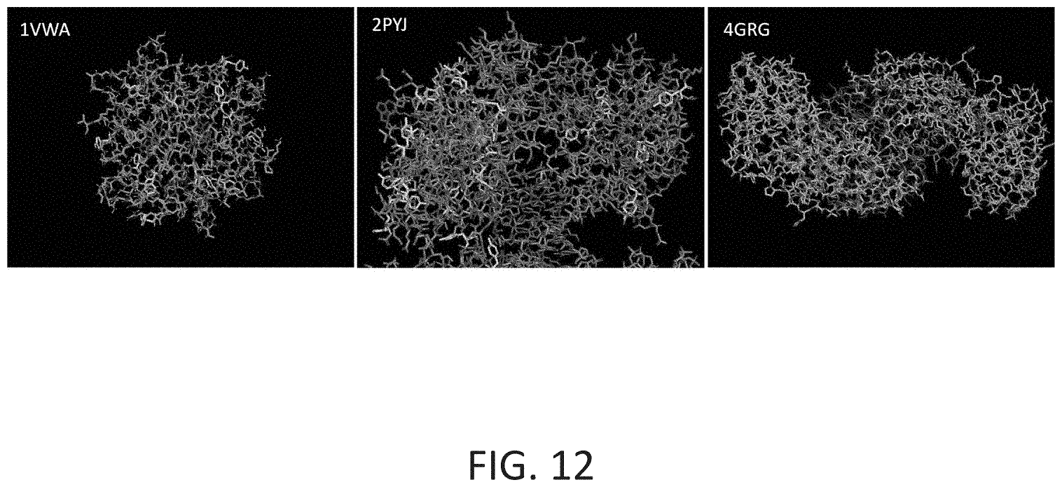

Although the specific chemical nature of the linker molecules alters the contact resistance, and hence overall conductance of the system, cyclic voltammetry shows that the linkers are not electroactive (as is also the case for the proteins described herein). Furthermore, the diverse nature of the chemical linkers is not compatible with the universal nature of the resonance demonstrated in the present disclosure. Thus, the resonance is most likely an intrinsic common feature of the proteins. The conduction path is through the protein: this is shown by experiments that compare the responses of IgG molecules with the corresponding Fab fragment, that measure the internal decay of conductance with distance, and that sense the changes in conductance as streptavidin binds biotin or as a polymerase binds a nucleotide triphosphate. This suggests that there may be a common feature of these proteins that accounts for the resonance. The closest redox potential among the amino acids are those for the oxidation of tyrosine and tryptophan at about 1000 to 1200 mV vs NHE (though the value can be lower, ˜500 mV, in deprotonated complexes). All three proteins contain many of these residues in their interiors (FIG. 12). Thus, the reduction of the Marcus reorganization energy barrier inside the protein (arising from non-ergodic sampling of electrostatic fluctuations proposed by Matyushov) could account for discrepancy between the redox potentials of these amino acids in solution and the conduction maximum energy in an intact protein.

Similar reductions in reorganization energy have been reported for accessible redox centers that are at least partially embedded in protein or where charge transfer is rapid. For example, the redox potential of transition metal aqua-ions is reduced significantly if these same ions are incorporated into a protein, and the energy loss for rapid electron transport for primary charge separation in bacterial photosynthesis is reduced to 0.25 eV compared to the equilibrium value of 1.4 eV. Although the values of the peak-conductance potentials are nearly the same in all three proteins (Table 2) one might expect the exact amount of reorganization energy to depend on atomic scale details, so the small differences observed may be significant. Deeper understanding of these effects requires detailed molecular modeling, and the streptavidin protein may be small enough to allow for calculations.

The observation of resonant tunneling (in the form of a resonance that fits the Breit-Wigner formula), and, in some proteins at least, long decay lengths and temperature independent conductance might appear to be consistent with conduction bands as proposed by Szent-Gyorgyi, but the possibility of long-lived quantum-coherence in proteins is controversial. However, theories that extend the Landauer formula to finite temperatures can explain all of these features without invoking coherent transport. In this modified Landauer approach, the Γ's in equation 3 represent coupling between the electrodes and the nearest energetically-available molecular orbital. In a simple single-tunneling barrier model the electronic coupling is exponentially related to a bond lifetime, so stronger coupling (i.e. larger Γ) should correlate with stronger bonding (or equivalently a smaller dissociation constant, K_{D}). K_{D} for the DNP-anti-DNP IgE bond is 65 nM (Γ=72 meV), and ˜10 fM for streptavidin-biotin (Γ=180 meV), qualitatively consistent with a relationship between bonding strength and electronic coupling (Table 2).

In accordance with the above, embodiments of the present disclosure include a bioelectronic device comprising a first electrode and a second electrode separated by a gap, and a protein attached to the first and second electrodes via a linker. In some embodiments, the electrical surface potential of the first and second electrodes at zero bias is from about 250 mV to about 400 mV on the normal hydrogen electrode scale. In some embodiments, the electrical surface potential of the first and second electrodes at zero bias is from about 260 mV to about 400 mV on the normal hydrogen electrode scale. In some embodiments, the electrical surface potential of the first and second electrodes at zero bias is from about 270 mV to about 400 mV on the normal hydrogen electrode scale. In some embodiments, the electrical surface potential of the first and second electrodes at zero bias is from about 280 mV to about 400 mV on the normal hydrogen electrode scale. In some embodiments, the electrical surface potential of the first and second electrodes at zero bias is from about 290 mV to about 400 mV on the normal hydrogen electrode scale. In some embodiments, the electrical surface potential of the first and second electrodes at zero bias is from about 250 mV to about 390 mV on the normal hydrogen electrode scale. In some embodiments, the electrical surface potential of the first and second electrodes at zero bias is from about 250 mV to about 380 mV on the normal hydrogen electrode scale. In some embodiments, the electrical surface potential of the first and second electrodes at zero bias is from about 250 mV to about 370 mV on the normal hydrogen electrode scale. In some embodiments, the electrical surface potential of the first and second electrodes at zero bias is from about 250 mV to about 360 mV on the normal hydrogen electrode scale. In some embodiments, the electrical surface potential of the first and second electrodes at zero bias is from about 250 mV to about 350 mV on the normal hydrogen electrode scale. In some embodiments, the electrical surface potential of the first and second electrodes at zero bias is from about 250 mV to about 340 mV on the normal hydrogen electrode scale. In some embodiments, the electrical surface potential of the first and second electrodes at zero bias is from about 250 mV to about 330 mV on the normal hydrogen electrode scale. In some embodiments, the electrical surface potential of the first and second electrodes at zero bias is from about 250 mV to about 320 mV on the normal hydrogen electrode scale. In some embodiments, the electrical surface potential of the first and second electrodes at zero bias is from about 250 mV to about 310 mV on the normal hydrogen electrode scale.

In some embodiments, conductance of the protein is maximized when the surface potential of the first and second electrodes at zero bias is from about 250 mV to about 400 mV. In some embodiments, conductance of the protein is maximized when the surface potential of the first and second electrodes at zero bias is from about 260 mV to about 400 mV. In some embodiments, conductance of the protein is maximized when the surface potential of the first and second electrodes at zero bias is from about 270 mV to about 400 mV. In some embodiments, conductance of the protein is maximized when the surface potential of the first and second electrodes at zero bias is from about 280 mV to about 400 mV. In some embodiments, conductance of the protein is maximized when the surface potential of the first and second electrodes at zero bias is from about 290 mV to about 400 mV. In some embodiments, conductance of the protein is maximized when the surface potential of the first and second electrodes at zero bias is from about 250 mV to about 390 mV. In some embodiments, conductance of the protein is maximized when the surface potential of the first and second electrodes at zero bias is from about 250 mV to about 380 mV. In some embodiments, conductance of the protein is maximized when the surface potential of the first and second electrodes at zero bias is from about 250 mV to about 370 mV. In some embodiments, conductance of the protein is maximized when the surface potential of the first and second electrodes at zero bias is from about 250 mV to about 360 mV. In some embodiments, conductance of the protein is maximized when the surface potential of the first and second electrodes at zero bias is from about 250 mV to about 350 mV. In some embodiments, conductance of the protein is maximized when the surface potential of the first and second electrodes at zero bias is from about 250 mV to about 340 mV. In some embodiments, conductance of the protein is maximized when the surface potential of the first and second electrodes at zero bias is from about 250 mV to about 330 mV. In some embodiments, conductance of the protein is maximized when the surface potential of the first and second electrodes at zero bias is from about 250 mV to about 320 mV. In some embodiments, conductance of the protein is maximized when the surface potential of the first and second electrodes at zero bias is from about 250 mV to about 310 mV.

In some embodiments, the first and second electrodes are comprised of a metal or metals that impart a surface potential from about 250 mV to about 400 mV at zero bias. In some embodiments, the first and second electrodes are comprised of a metal or metals that impart a surface potential from about 260 mV to about 400 mV at zero bias. In some embodiments, the first and second electrodes are comprised of a metal or metals that impart a surface potential from about 270 mV to about 400 mV at zero bias. In some embodiments, the first and second electrodes are comprised of a metal or metals that impart a surface potential from about 280 mV to about 400 mV at zero bias. In some embodiments, the first and second electrodes are comprised of a metal or metals that impart a surface potential from about 290 mV to about 400 mV at zero bias. In some embodiments, the first and second electrodes are comprised of a metal or metals that impart a surface potential from about 250 mV to about 390 mV at zero bias. In some embodiments, the first and second electrodes are comprised of a metal or metals that impart a surface potential from about 250 mV to about 380 mV at zero bias. In some embodiments, the first and second electrodes are comprised of a metal or metals that impart a surface potential from about 250 mV to about 370 mV at zero bias. In some embodiments, the first and second electrodes are comprised of a metal or metals that impart a surface potential from about 250 mV to about 360 mV at zero bias. In some embodiments, the first and second electrodes are comprised of a metal or metals that impart a surface potential from about 250 mV to about 350 mV at zero bias. In some embodiments, the first and second electrodes are comprised of a metal or metals that impart a surface potential from about 250 mV to about 340 mV at zero bias. In some embodiments, the first and second electrodes are comprised of a metal or metals that impart a surface potential from about 250 mV to about 330 mV at zero bias. In some embodiments, the first and second electrodes are comprised of a metal or metals that impart a surface potential from about 250 mV to about 320 mV at zero bias. In some embodiments, the first and second electrodes are comprised of a metal or metals that impart a surface potential from about 250 mV to about 310 mV at zero bias.

In some embodiments, at least one of the first and second electrodes comprises a different metal as that of the other electrode. In some embodiments, at least one of the first and second electrodes comprises gold or an alloy thereof. In some embodiments, both the first and second electrodes comprise gold or an alloy thereof. In some embodiments, the first electrode comprises gold or an alloy thereof and the second electrode comprises a different metal or an alloy thereof. In some embodiments, the second electrode comprises palladium or an alloy thereof. In some embodiments, the second electrode comprises platinum or an alloy thereof.

In some embodiments, the device comprises a reference electrode. In some embodiments, the surface potential of the first and second electrodes is maintained at about 250 mV to about 400 mV on the NHE scale due to a bias applied between the reference electrode and at least one of the first or second electrode. As would be recognized by one of ordinary skill in the art based on the present disclosure, applying a fixed bias between a given reference electrode and another metal will generate a reproducible polarization at the surface of that second metal. Therefore, a surface potential of an electrode pair can be selected (e.g., by selecting metals and/or biasing with respect to a reference electrode), for which zero bias is initially applied across the electrode pair. Then, application of a bias across the electrode pair will shift the surface potential of the biased electrode by the amount of the applied bias. So, if, for example it is desired to hold the average potential of the pair at 300 mV on the NHE scale with a bias of +100 mV applied, a first electrode can be set to a potential of 250 mV on the NHE scale, so that with the +100 mV bias applied to the second electrode with respect to the first, the second electrode is at +350 mV on the NHE scale, so that the average of the two electrode potentials is the desired 300 mV on the NHE scale.

In some embodiments, the surface potential of the first and second electrodes is maintained at about 250 mV to about 400 mV due to a bias applied between the reference electrode and at least one of the first or second electrode. In some embodiments, the surface potential of the first and second electrodes is maintained at about 260 mV to about 400 mV due to a bias applied between the reference electrode and at least one of the first or second electrode. In some embodiments, the surface potential of the first and second electrodes is maintained at about 270 mV to about 400 mV due to a bias applied between the reference electrode and at least one of the first or second electrode. In some embodiments, the surface potential of the first and second electrodes is maintained at about 280 mV to about 400 mV due to a bias applied between the reference electrode and at least one of the first or second electrode. In some embodiments, the surface potential of the first and second electrodes is maintained at about 290 mV to about 400 mV due to a bias applied between the reference electrode and at least one of the first or second electrode. In some embodiments, the surface potential of the first and second electrodes is maintained at about 250 mV to about 390 mV due to a bias applied between the reference electrode and at least one of the first or second electrode. In some embodiments, the surface potential of the first and second electrodes is maintained at about 250 mV to about 380 mV due to a bias applied between the reference electrode and at least one of the first or second electrode. In some embodiments, the surface potential of the first and second electrodes is maintained at about 250 mV to about 370 mV due to a bias applied between the reference electrode and at least one of the first or second electrode. In some embodiments, the surface potential of the first and second electrodes is maintained at about 250 mV to about 360 mV due to a bias applied between the reference electrode and at least one of the first or second electrode. In some embodiments, the surface potential of the first and second electrodes is maintained at about 250 mV to about 350 mV due to a bias applied between the reference electrode and at least one of the first or second electrode. In some embodiments, the surface potential of the first and second electrodes is maintained at about 250 mV to about 340 mV due to a bias applied between the reference electrode and at least one of the first or second electrode. In some embodiments, the surface potential of the first and second electrodes is maintained at about 250 mV to about 330 mV due to a bias applied between the reference electrode and at least one of the first or second electrode. In some embodiments, the surface potential of the first and second electrodes is maintained at about 250 mV to about 320 mV due to a bias applied between the reference electrode and at least one of the first or second electrode. In some embodiments, the surface potential of the first and second electrodes is maintained at about 250 mV to about 310 mV due to a bias applied between the reference electrode and at least one of the first or second electrode.

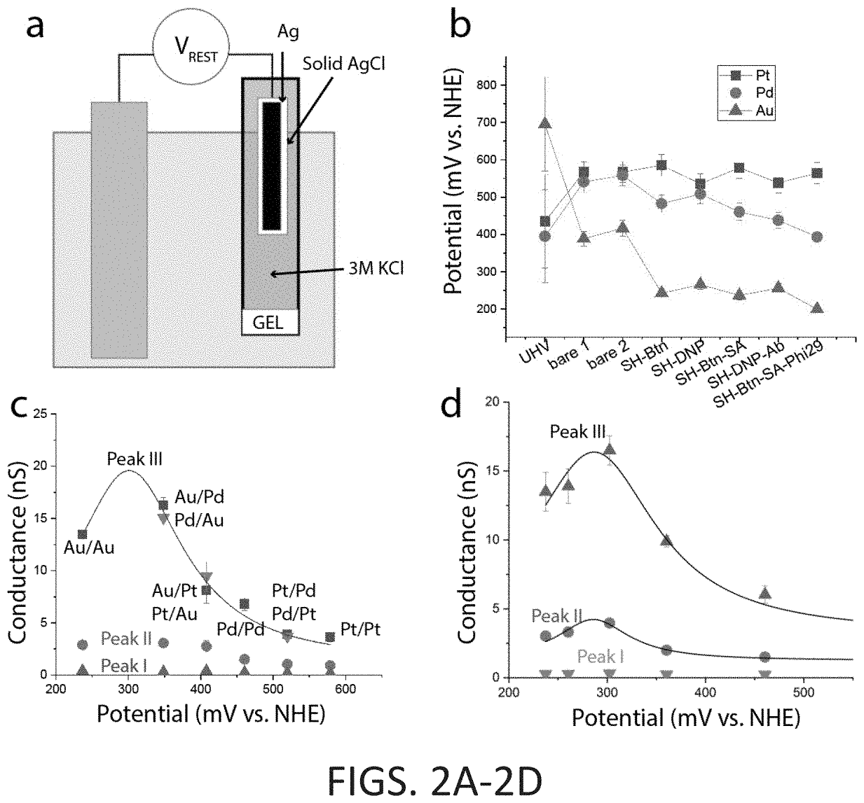

In some embodiments, and as further illustrated in FIG. 2A, the reference electrode can include a third electrode immersed in an electrolyte solution that is in contact with the first and second electrodes. The electrolyte solution can be any suitable electrolyte solution used to conduct electricity (e.g., potassium chloride, sodium phosphate, sodium biphosphate, etc.), as would be recognized by one of ordinary skill in the art based on the present disclosure. Other reference electrode configurations can also be used.

Device Composition and Geometry. Gold is the most widely-used metal in molecular electronic devices, partly because it is relatively easy to make high-quality molecular monolayers on gold and partly because it is used to make molecular break-junctions, the most common method for mounting molecules in an electrode junction. Both advantages rely on the malleability and low melting point of gold. Good monolayers form because a thiol bond between a molecule and the metal weakens the bonding of the attached gold atom to its neighbors to the point where the gold atom, with the molecule attached, can move on the surface quite freely, allowing for dense packing of a monolayer. In the case of the break-junction technique, a new junction is extruded on each approach/retraction cycle of the electrode pair, a consequence of the malleability of the gold. However, this malleability also presents challenges for device fabrication. If a gap is formed by two adjacent gold features on an integrated circuit, the metal at the edges of the feature can be quite mobile, so the dimensions of the junction are not stable at the atomic level. For this reason, noble metals with a higher melting point are to be preferred in device fabrication. However, gold electrodes have the added advantage of having a Fermi level that is well matched to the energy levels of many candidate molecular devices, including particularly protein-based devices. Accordingly, there exists a need for a junction device composition and geometry that provides the shape stability of a noble metal with a higher melting point, but with the electronic properties of gold.

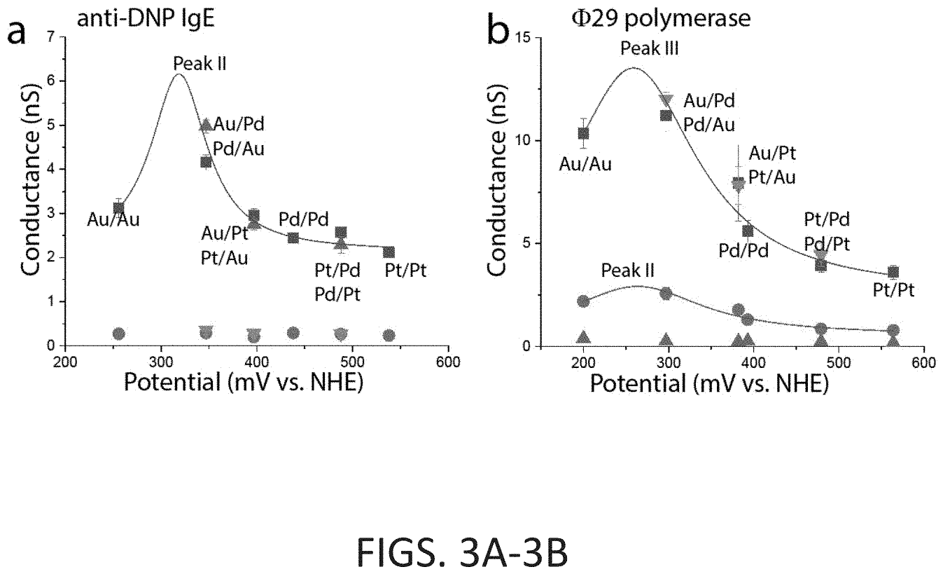

As described further herein, distributions of conductance were measured for molecular junctions in which a streptavidin molecule bridges two metal electrodes functionalized with thiolated biotin molecules. Using different metals, including alloys thereof, for the contacts, conductance of a protein-of-interest can be maximized. For example, FIG. 3 shows measured single molecule conductances for six different combinations of metal. In the case of mixed metal junctions, the first listed metal refers to the tip in a scanning probe microscope and the second to the substrate. Measurements were repeated with the tip and substrate metals reversed as shown (e.g., Au/Pt vs Pt/Au). Thus, while platinum electrodes are to be preferred in terms of stability and resistance to oxidation, gold is clearly superior in terms of electronic response. In some embodiments, the use of different metals is to be preferred for the contacts, and in some cases, Pd/Au and Pt/Au is particularly useful. In the devices of the present disclosure, the use of a combination of metals circumvents the problems posed by the unstable edges of gold electrodes.

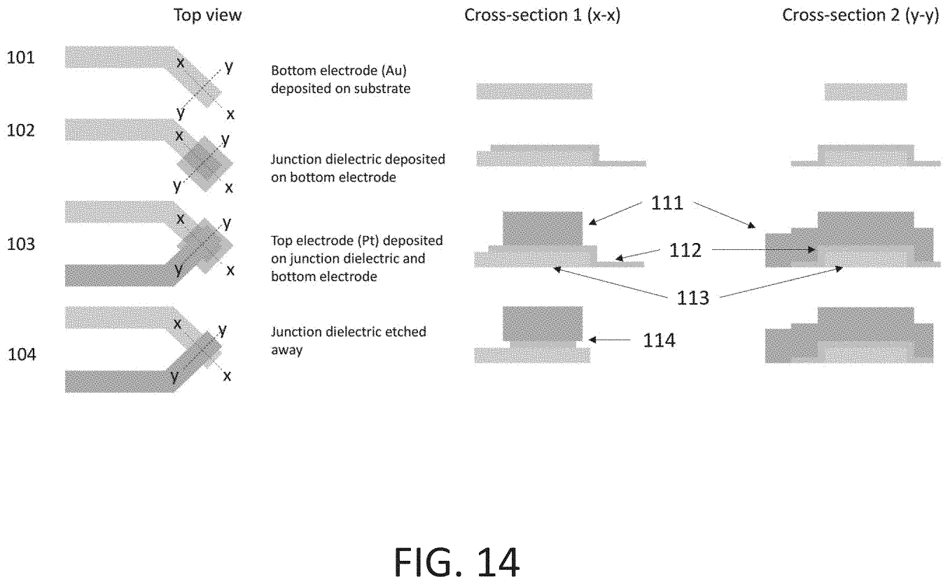

Referring to FIG. 14, a gold electrode, 101, is first deposited on a dielectric substrate. The substrate may be any dielectric material such as glass or quartz. The dielectric substrate may be a layer of dielectric insulation. Alternately, the substrate can be a high-resistivity silicon with a thick (about 500 nm) layer of oxide grown on it. The gold electrode may be patterned according to methods known in the art, such as by standard lift-off methods. If a dual layer of photoresist is used so as to allow for an undercut mask, the edges of the gold electrode can be free of fencing asperities. In some embodiments, if the gold is deposited at an angle onto a rotating substrate with an undercut photoresist mask, the edges can be made to be gently sloping. The electrode 101 can be from about 50 nm to about 20 μm wide and from about 5 nm to about 1 μm thick.

In some embodiments, a dielectric 102 is deposited over one end of the gold electrode using standard photolithographic methods followed by atomic layer deposition (ALD). This dielectric may be SiO₂, HfO₂, Al₂O₃ or any other dielectric material that can be reliably deposited as a thin film using atomic layer deposition. Typically, the amount of dielectric deposited is from about 1 nm to about 50 nm. Improved ALD growth of very thin films is obtained by treating the surface of the first electrode (e.g., a planar electrode, a bottom electrode) with a very thin (about 1 nm or less) layer of a reactive metal such as Cr, Ti or Al.

In some embodiments, a second electrode 103 is deposited so as to lie over the top of dielectric-coated first electrode, as shown in the cross-sections to the right: 113 is the first gold electrode (positioned on the bottom, on top of the substrate), dielectric layer 112 and the second electrode 111. The second electrode can be any noble metal. In some embodiments, the second electrode is made from platinum or palladium. The second electrode may be from about 50 nm to about 10 μm in width and from about 5 nm to about 100 nm thick. In determining the width of the second electrode, the constraint is that the edges of the second electrode lie over a planar portion of the first electrode.

In some embodiments, the dielectric is then etched away from the first electrode using a slow, wet-etchant, such as buffered HF (typically a solution of HF and NH₄F), piranha solution (H₂SO₄ and H₂O₂) and/or a HCl/H₂O₂ solution for HfO₂ dielectric layers and SiO², and Tetramethylammonium hydroxide (TMAH) or a similar base like KOH for Al₂O₃ dielectric layers. The amphoteric nature of the last atomic layer of oxide deposition can result is resistance to basic etches, and an added acids wash improves completeness of the layer removal. The result is a slight undercutting of the dielectric under the junction as shown by 114 on FIG. 14.

In some embodiments, covering of the edge of the gold electrode with dielectric confers certain advantageous characteristics, for example, preventing motion of the edge atoms of the gold electrode. By using a more stable metal (e.g., Pd, Pt) for the second electrode, the edge of the second electrode defines a sharp junction with respect to the underlying planar gold surface. Additionally, the avoidance of RIE or other particle-bombardment methods to expose a junction as used in some earlier designs of layered junction devices can be an important consideration.

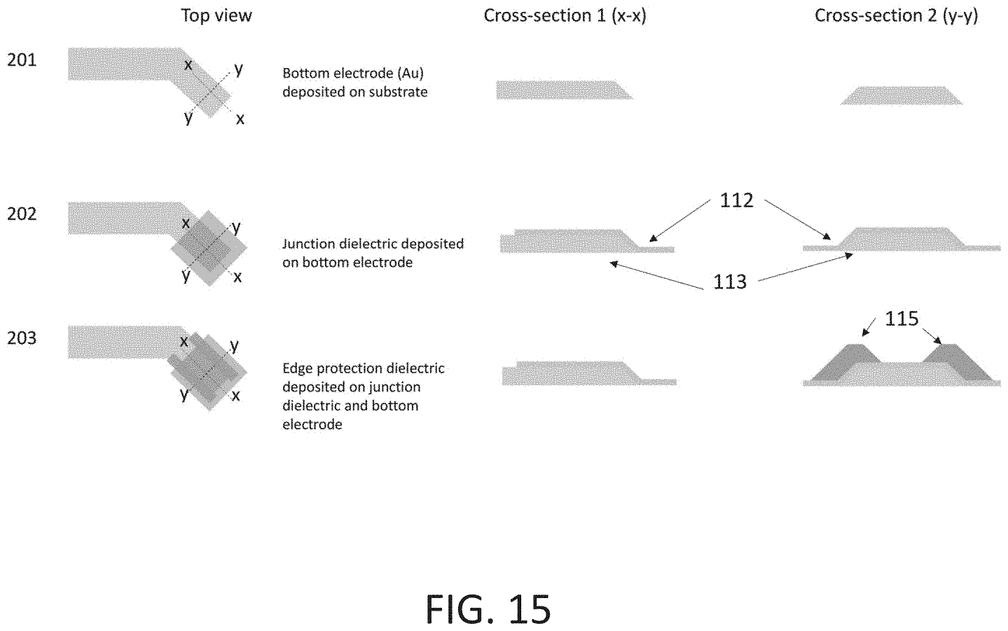

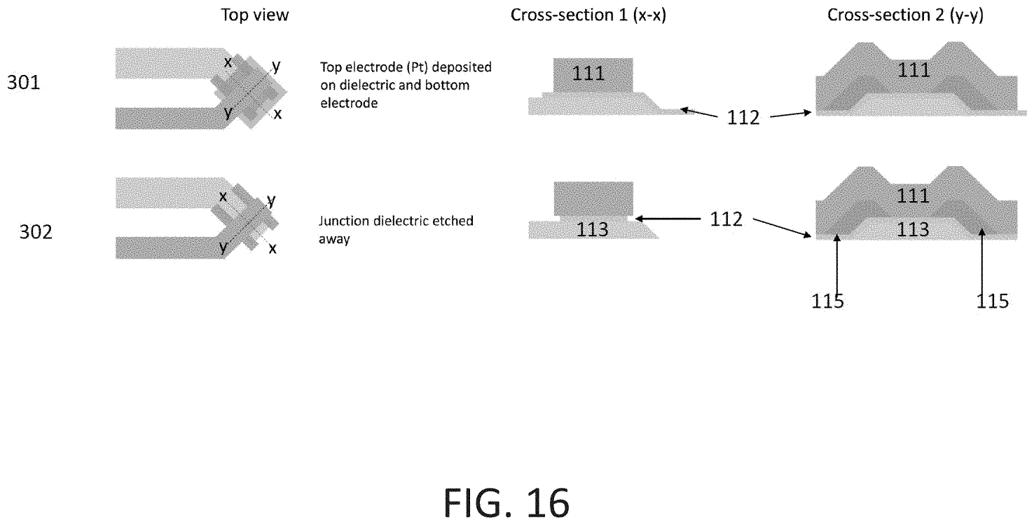

In some embodiments, it may be desirable to incorporate further protection at the edge of the first gold electrode. A scheme for this is shown in FIG. 15. In some embodiment, a first gold electrode 201 is formed on top of a substrate, and covered with dielectric 202 as described above. In some embodiments, a second layer of dielectric 115 is patterned over the edges of the first gold electrode as shown in 203. Referring to FIG. 16, the second Pd or Pt electrode 111 is then formed over the junction as shown in 301. Etching of the dielectric layer 112 clears the middle part of the junction, but leaves the edges protected by the additional dielectric 115.

Additionally, the entire device may be passivated using, for example a layer of SU8 polymer of about 500 nm to about 15 μm thickness, opened to expose the junction in a small window of a few microns on each side. An alternative is about 50 nm to about 200 nm thick layer of HfO₂, Al₂O₃ or SiO₂, preferably deposited by atomic layer deposition.

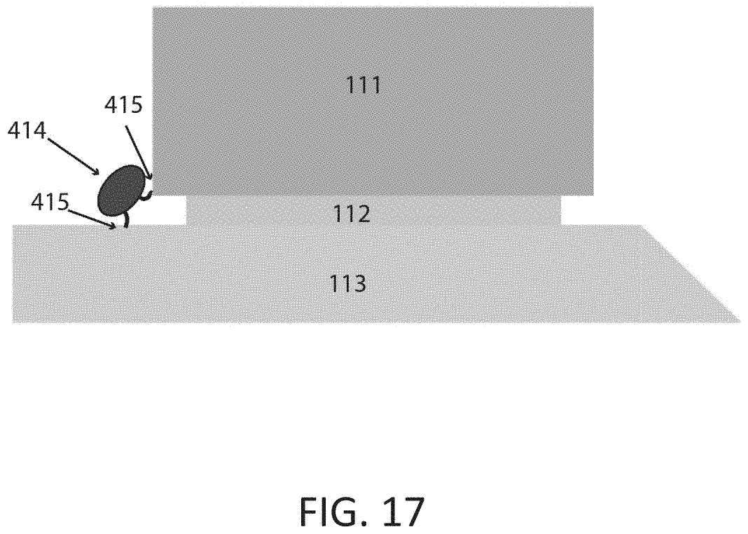

Once the window is opened, the molecular junction may be further cleaned by exposure to an oxygen plasma and functionalized with molecules. The result is a molecular junction as shown in FIG. 17. The second electrode 111 and first electrode 113 are each functionalized with a ligand 415 that traps the protein to be incorporated 414 across the j unction.

The ligands used in the devices and systems described herein can be specific for a protein and are modified so that they attach to the electrodes. For example, the ligand can be modified to contain a thiol termination at one end for coupling to metals. Examples of ligands are peptide epitopes for antibodies comprising a cysteine residue at one end, recognition peptides (e.g., such as the RGD peptide for binding integrin comprising a cysteine) and small molecules to which proteins have been selected to bind (e.g., such as an IgE molecule that binds dintitrophenyl and comprising a thiol, or a thiolated biotin molecule). The various configurations and geometries of the bioelectronic devices of the present disclosure can include any aspects of the devices disclosed in U.S. Pat. No. 10,422,787 and PCT Appln. No. PCT/US2019/032707, both of which are herein incorporated by reference in their entirety and for all purposes.

In some embodiments, one of ordinary skill in the art will recognize that since the work function of metal alloys is generally given by a weighted average of the work functions of their component metals, gold alloys may be substituted for the first electrode. For example, white gold (alloys with palladium and or silver) and other gold alloys such as with copper or nickel may be used in place of pure gold. Similarly, alloys can be used for the second electrode, such as palladium-platinum, palladium-silver, platinum-silver and others.

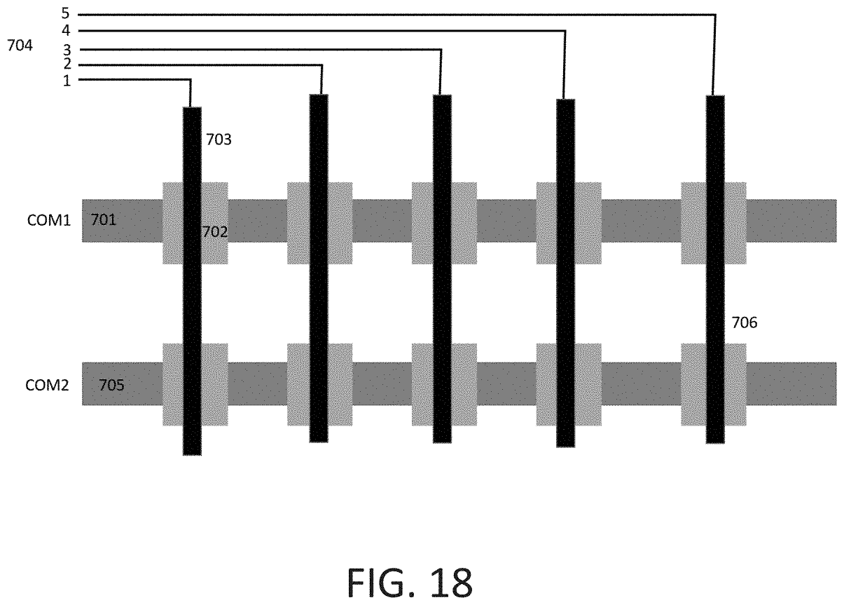

The junction design of the present disclosure lends itself to multiplexed addressing of an array of junction devices, as illustrated in FIG. 18. While FIG. 18 shows an array of 10 devices, it will be understood by one of ordinary skill in the art based on the present disclosure that an array can comprise hundreds or even thousands of junctions. Each device can be separately functionalized with a given ligand, so that the array can test for the presence of many different protein at one time.

Referring to FIG. 18, the first electrode can form a common connection for an array of devices (com1, 703, com2, 705). Additionally, the dielectric layer 702 can patterned at each location in which a junction is to be formed. Second electrodes 703 can then be deposited so as to cross as many common electrodes as desired (shown here for just two: com1 and com2). Each second electrode is individually addressed. In a dense array, this addressing can be achieved via multiplexing electronics associated with each block of devices corresponding to the capacity of the multiplexing electronics. FIG. 18 shows 5 address lines, labeled 1-5 (704). Thus, for example, the device 706 is addressed by com2 and address line 5.

In some embodiments, the gap has a width of about 1.0 nm to about 20.0 nm. In some embodiments, the first and second electrodes are separated by a dielectric layer, as described further herein.

In some embodiments, the protein is a non-redox protein. In some embodiments, the protein includes, but is not limited to, a polymerase, a nuclease, a proteasome, a glycopeptidase, a glycosidase, a kinase and an endonuclease.

In some embodiments, the linker is attached to an inactive region of the protein. In some embodiments, the linker comprises a covalent chemical bond. In some embodiments, the protein is biotinylated. In some embodiments, the linker comprises thio-streptavidin. In some embodiments, the protein and the first and second electrodes are biotinylated, and wherein the linker comprises a streptavidin molecule comprising at least two biotin binding sites.

Embodiments of the present disclosure also include a system for direct electrical measurement of protein activity. In accordance with these embodiments, the system includes any of the bioelectronic devices described herein, a means for introducing an analyte capable of interacting with the protein, a means for applying a voltage bias between the first and second electrodes that is 100 mV or less, and a means for monitoring fluctuations that occur as the chemical entity interacts with the protein.

Embodiments of the present disclosure also include an array comprising a plurality of any of the bioelectronic devices described herein. In some embodiments, the array includes a means for introducing an analyte capable of interacting with the protein, a means for applying a voltage bias between the first and second electrodes that is 100 mV or less, and a means for monitoring fluctuations that occur as the chemical entity interacts with the protein. The array can be configured in a variety of ways, as exemplified in FIG. 18, which is not to be taken as limiting.

Embodiments of the present disclosure also include a method for direct electrical measurement of protein activity. In accordance with these embodiments, the method includes introducing an analyte capable of interacting with the protein to any of the bioelectronic devices described herein, applying a voltage bias between the first and second electrodes that is 100 mV or less, and observing fluctuations in current between the first and second electrodes that occur when the analyte interacts with the protein. In some embodiments, the analyte is a biopolymer, such as, but not limited to, a DNA molecule, an RNA molecule, a peptide, a polypeptide, or a gly can.

### 3. MATERIALS AND METHODS

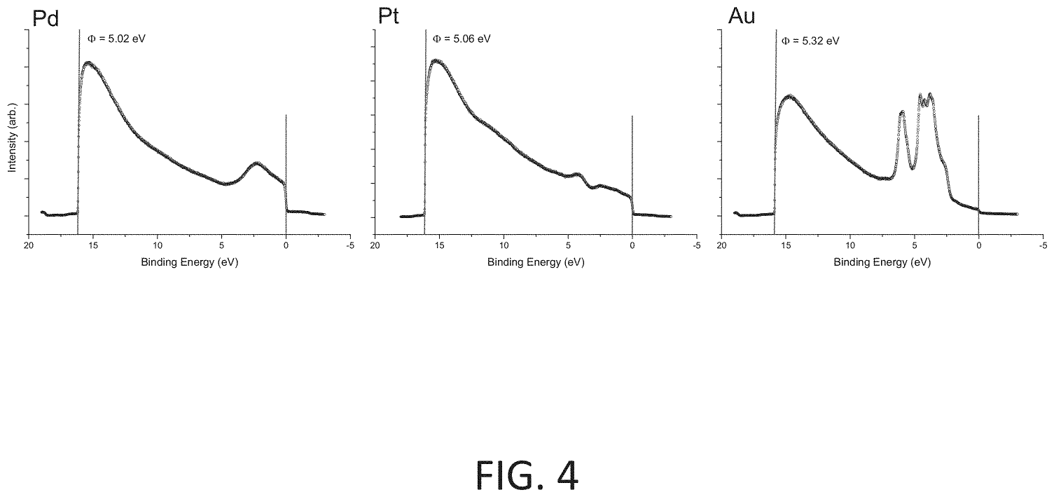

Approximately 200 nm of Pd, Au or Pt were deposited onto a 10 nm Cr adhesion layer on one inch p-type Si wafers using an e-beam evaporator (Lesker PVD 75). Samples were cleaned in an electron cyclotron resonance microwave plasma chemical vapor deposition (ECR-CVD) system using mixture of H₂ (20 sccm) and Ar (2.5 sccm). Samples were transported via an UHV transfer line (5·10⁻⁹ Torr) from the ECR-CVD to a photoelectron spectroscopy chamber equipped with a differentially pumped helium discharge lamp (21.2 eV) for ultraviolet photoemission spectroscopy (UPS) with a working pressure of ˜4-8·10⁻⁹ Torr. An Omicron Scientia R3000 hemispherical analyzer operated with a pass energy of 2 eV corresponding to an energy resolution of 3 meV. A sample bias of 1.5V and energy offset of 2.7 eV is programmed into the data acquisition software to compensate for the detector work function (4.2 eV). Fits to the UPS spectra are shown in FIG. 4 and a summary of work functions measured before and after cleaning is given in Table 3.

For the electrochemical measurements, salt-bridged electrodes were constructed as described previously using 3M KCl for the rest potential measurements (210 mV on the NHE scale) and 10 mM KCl for the conductance measurements (360 mV on the NHE scale). Rest potentials were measured with a Fluke 177 meter (input impedance >10⁷Ω) and potentials were stable to within ±5 mV over a period of hours. Sample to sample variation was ±5%.

High density polyethylene-coated Pd and Au probes were prepared as described previously. For Pt probe preparation, a home-made etching controller was used, outputting an AC voltage of 30 V with a frequency of about 250 Hz. The etching solution for Pt probes was freshly prepared 10 M NaOH.

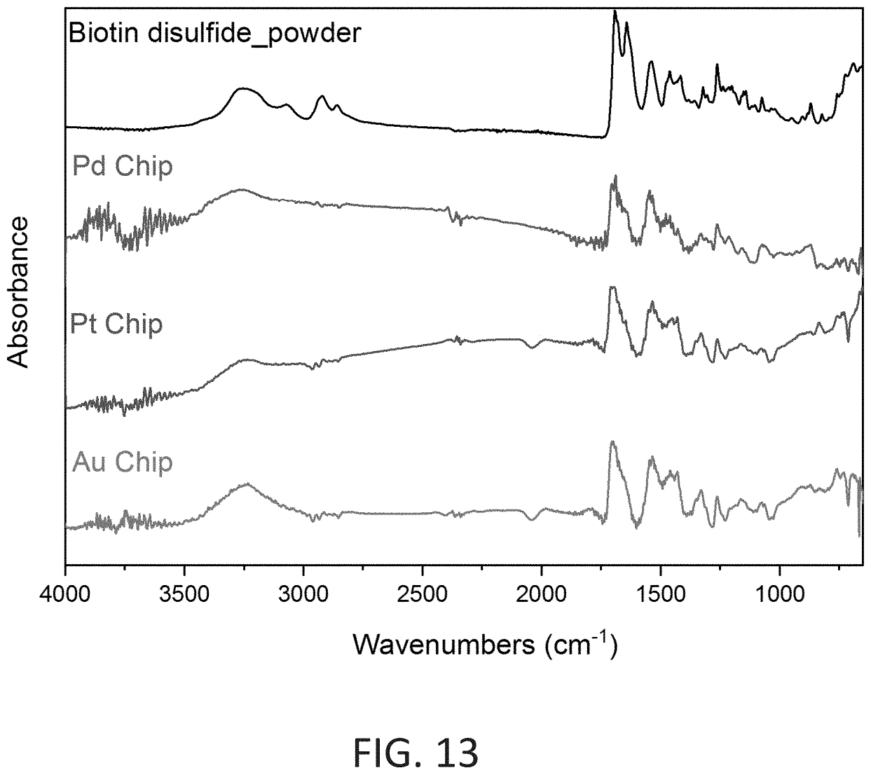

Substrates were prepared as described above, and functionalized as previously described. Conductance measurements were made in 1 mM phosphate buffer, pH 7.4, using a PicoSPM (Agilent) following the procedure described elsewhere. Samples and solutions were prepared as described earlier for biotin-streptavidin and for the biotin-streptavidin-polymerase Φ29 system, using a doubly-biotinylated engineered polymerase. The preparation of all solutions, and characterization of substrate surfaces is also described in these earlier publications. FTIR spectra taken from all three metal substrates are given in FIG. 13.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Controlling electron injection energy by changing the electrode metal. The calculated HOMO-LUMO gaps of most proteins are large, so that the Fermi energy of a metal electrode should be far from that of molecular orbital energies if the Fermi level was located at mid-gap. However, interfacial polarization (and hence the location of molecular orbitals relative to metal Fermi energies) is difficult to calculate, so a robust method is needed to measure these energies. The energy of molecular states responsible for transport can be probed by measuring the conductance of molecules with different electrode metals. In these prior studies, the metal work function served as a measure of the electronic injection energy. This approximation should not hold generally, because the surface potential of an electrode is extremely sensitive to chemical modification. Accordingly, the measurements reported here are carried out under electrochemical potential control, so that the rest potentials of modified surfaces can be used to quantify the changes in potential as surfaces are chemically modified.

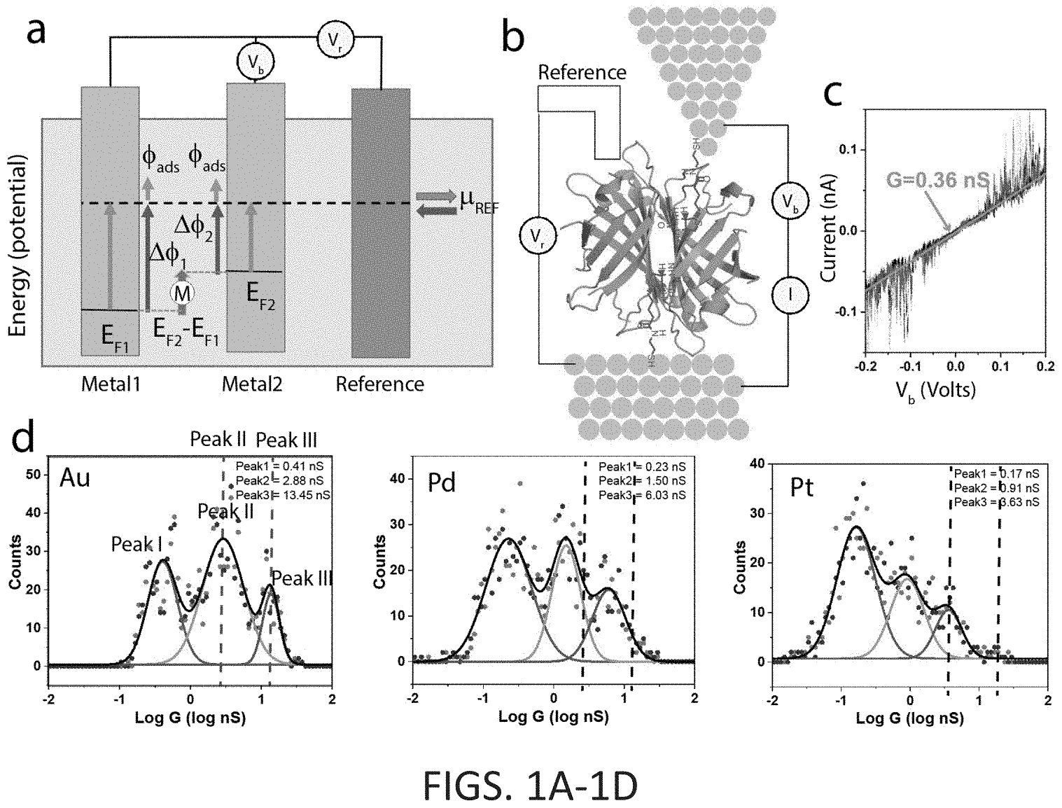

The experimental arrangement is illustrated schematically in FIG. 1A. A first electrode (Metal 1) is held at a potential Vᵣ with respect to the reference electrode. A second electrode (Metal 2) is held at a potential V_{b} with respect to Metal 1. The molecule (M) sits in a nanoscale gap between Metal 1 and Metal 2. The potential of an electron when it passes on to the molecule from one of the electrodes was investigated. The case where both the reference bias, Vᵣ, and the molecular junction bias, V_{b}, are zero were initially considered. The Fermi level of the reference electrode is pinned at the redox potential of the redox couple in solution, μ_{REF} by Faradaic processes that maintain constant polarization of the reference electrode surface. The reference, in turn, supplies or withdraws carriers from each of the metal electrodes (via low impedance connections) so as to move their Fermi levels, E_{F1} and E_{F2} into alignment at the energy μ_{REF}. The work function is defined by Φ=ϕ−E_{F} where ϕ is the rise in mean electrostatic potential across the metal surface, generated by the surface dipole (energies are expressed in eV). Accordingly, when the bulk electrochemical potential is changed from E_{F} to that of the reference, μ_{REF}, the change in ϕ is Δϕ=μ_{REF}−E_{F}. The potential difference seen by a carrier passing from the electrode to a molecule outside the electrode is given by Δϕ+ϕ_{ads} where ϕ_{ads} is the potential difference across an adlayer (which is assumed to be the same for both electrodes here). If the two electrode metals are not the same, then the net potential difference between the electrodes is Δϕ₁−Δϕ₂=μ_{REF}−E_{F1}−(μ_{REF}−E_{F2})=E_{F2}−E_{F1}. If the molecule is assumed to sit in the middle of the electric field generated by this difference, then the total potential difference that a carrier experiences in moving from electrode 1 to the molecule is:

$$Δ V = {E}_{F 1} + {ϕ}_{ads} - \frac{{E}_{F 1} - {E}_{F 2}}{2} = \frac{{E}_{F 2} + {E}_{F 1}}{2} + {ϕ}_{ads} \qquad ( 1 )$$

with an identical expression for the case of a carrier moving from electrode 2 to the molecule. When only one electrode material is used, equation 1 becomes

ΔV₁=E_{F1}+ϕ_{ads} (2)

This quantity is the rest potential—the potential difference between the modified metal and the reference measured at infinite impedance (these potentials were translated to those referred to as the Normal Hydrogen Electrode (NHE)). For two different electrode metals, the average of the two rest potentials yields the right hand side of equation 1, and thus the potential difference experienced as a carrier moves from either electrode to the middle of the gap. For the case of two identical metal electrodes, this difference is given by equation 2.

Rest potentials were measured relative to a 3M Ag/AgCl reference (FIG. 2A) using a high impedance voltmeter. Substrates were prepared by sputtering 205±5 nm of Pt, Pd and Au onto a silicon substrate coated with a 10 nm Cr adhesion layer. Ultraviolet Photoemission Spectroscopy (UPS, FIG. 4; Table 3) was used to determine the work functions as 5.32 eV (Au), 5.02 eV (Pd) and 5.06 eV (Pt), values that are shown on FIG. 2B as the points labeled UHV (Ultra High Vacuum). They have been converted to mV vs. NHE using the value of 4.625±0.125 eV for the work function of the NHE (measurements were accurate to within a few percent—the error bars show the uncertainty in the NHE work function). These values change dramatically on contact with the 1 mM phosphate buffer used for conductance measurements (labeled ‘bare 1’ and ‘bare 2’, where two measurements on different samples are shown to illustrate the ±5% reproducibility). Subsequent modifications (Table 1; FIG. 2B) have little effect on Pt, a small effect on Pd and a large effect on Au surfaces.

TABLE 1
Rest potentials measured vs an Ag/AgC1 reference with a 3M KC1 bridge,
converted to NHE by adding 210 mV. ¹UHV data were measured to ±4 meV: the error quoted
here (125 meV) represents the spread of values currently accepted for the work function of the
NHE. ²Errors reflect stability of rest potential measurement. Repeat measurement (see bare
chip repeat) indicates a run-to-run variation of ±5% (the error bars used in FIG. 2B).

| | | Pt (mV vs | Pd(mV vs | Au(mV vs |
|---|---|---|---|---|
| | Description | NHE) | NHE) | NHE) |
| UHV¹ | Plasma-cleaned films in UHV | 435 ± 125¹ | 395 ± 125 | 695 ± 125 |
| Bare chip | Films under 1 mM | 566.8 ± 3.8² | 540.7 ± 17 | 388.4 ± 4.3 |
| 1 mM PB | phosphate buffer | | | |
| Chip/SH-btn | Functionalized with | 585.3 ± 0.2 | 482.3 ± 0.1 | 242.5 ± 0.7 |
| | thiolated biotin | | | |
| Chip/SH-btn/SA | As above bound by streptavidin | 578.9 ± 0.4 | 460.5 ± 1.9 | 236.8 ± 0.6 |
| Chip/SH- | As above bound by a doubly- | 564.4 ± 2.8 | 393.4 ± 0.2 | 199.8 ± 2.8 |
| btn/SA/029 | biotinylated ϕ29 polymerase | | | |
| Bare chip | Films under 1 mM | 567.6 ± 3.3 | 558.5 ± 1.8 | 417.2 ± 1.8 |
| (repeat) | phosphate buffer | | | |
| Chip/SH-DNP | Functionalized with | 534.0 ± 4.1 | 508.3 ± 0.7 | 266.4 ± 1.0 |
| | thiolated DNP | | | |
| Chip/SH-DNP/Ab | As above + anti-DNP IgE | 537.8 ± 0.7 | 438.1 ± 0.3 | 256.1 ± 0.2 |

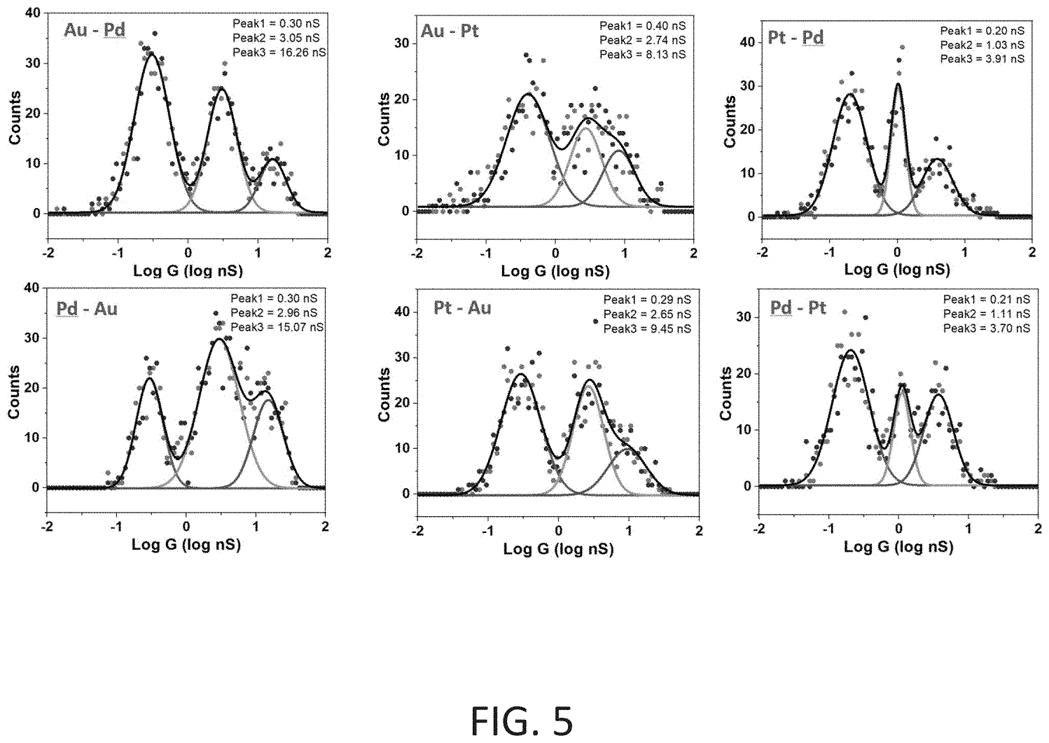

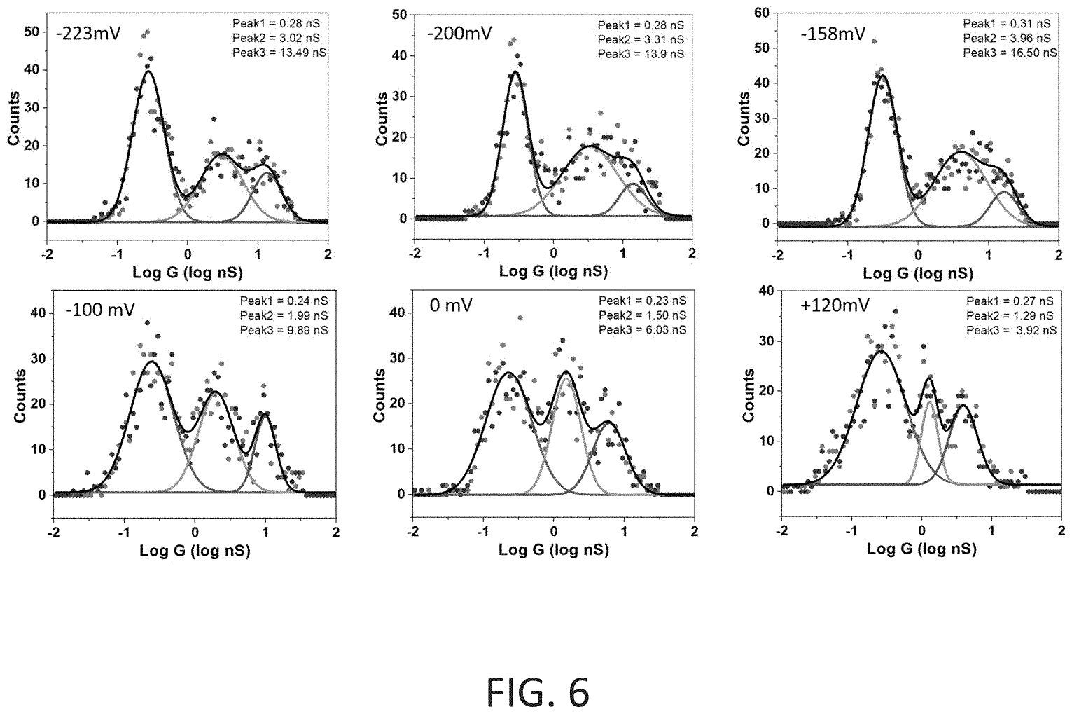

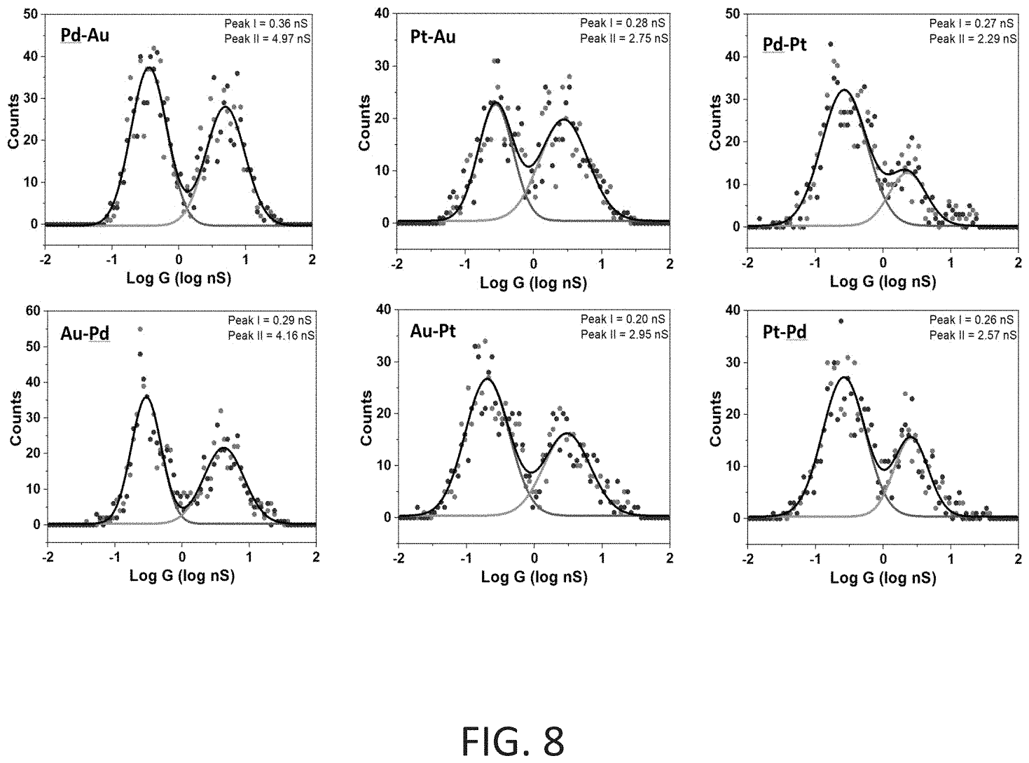

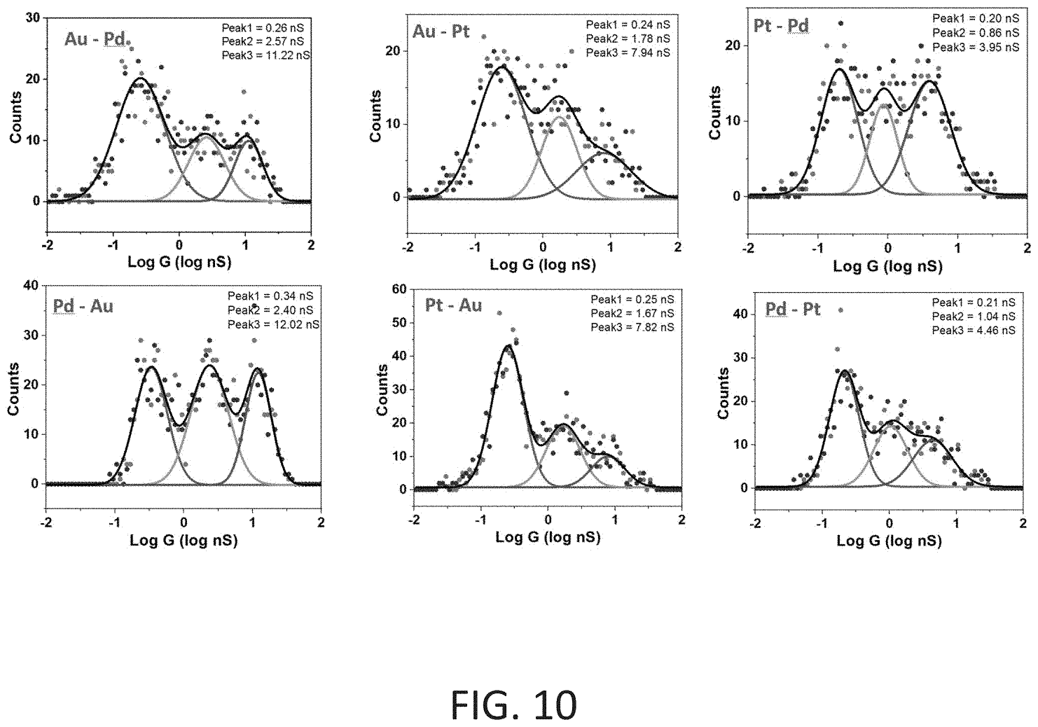

Conductance measurements were made by recording IV curves using an STM with a fixed gap and electrodes functionalized with ligands to trap the target proteins. The first system studied was streptavidin bound to electrodes functionalized with a thiol-terminated biotin (FIG. 1B) for which the gap was set to 2.5 nm. Trapped proteins gave perfectly linear current-voltage curves, displaying characteristic telegraph noise above ±100 mV (FIG. 1C). Many repeated measurements of the gradient of these curves yield conductance distributions for all the contact geometries sampled, examples of which are shown for the three metals in FIG. 1D. Contact resistance is smallest for the two higher conductance peaks (labeled peaks II and III), so it was assumed that these are the most sensitive to the internal electronic properties of the molecule. Both peaks move to lower conductance in going from Au to Pd electrodes, and to even lower conductance in going from Pd to Pt, illustrating the sensitivity of the conductance to electron energy, even in this non-redox active protein. These measurements were repeated with mixed electrode combinations (Au/Pd, Pd/Pt, Au/Pt) to obtain data points at three additional potentials (using potentials calculated with equation 1). The metals used for the tip and substrate were also reversed, finding that the conductance peak values were unaltered (though the height of peaks II and III changed a little, probably because of the more facile and mobile thiol bonding on Au substrates). Conductance distributions for all experiments are given in FIGS. 5-10 and the parameters extracted from Gaussian fits to these distributions are given in Tables 3-8. Results for the biotin-streptavidin junctions are summarized in FIG. 2C. The data points for peak III have been fitted with a Lorentzian (as described in the discussion) to yield a peak at a potential of 301±3 mV vs NHE (with a full width at half maximum, FWHM, of 183±43 mV).

### Example 2

Controlling electron injection energy by changing the electrode metal. In the case of Pd electrodes, the region of potential free of Faradaic currents is large enough to allow for testing of the resonance curve by varying the electrode potential (Vᵣ in FIG. 1A) in which case the carrier energy is given by adding Vᵣ to the rest potential given by equation 2. The results of these measurements are shown for the biotin-streptavidin system in FIG. 2D. The resonance for Peak III is fitted by an essentially identical Lorentzian to that used in FIG. 2C for the case of different metals, with a maximum at 287±8 mV vs. NHE and a FWHM of 154±28 mV. The agreement between the two methods validates the assumptions used in the model for the changes in potential experienced with the different electrode metals.

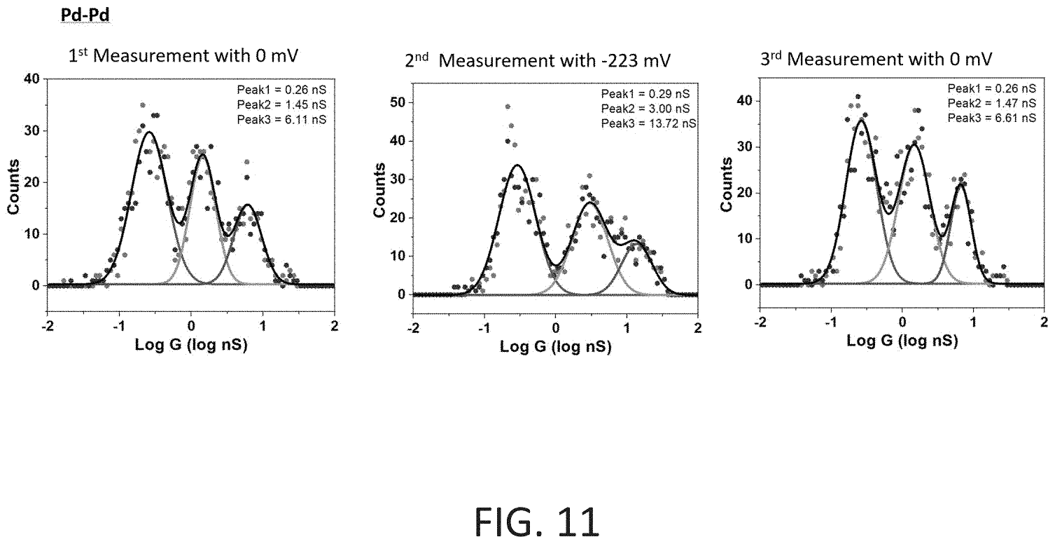

As a check of reversibility, a separate set of experiments was run in which a sample was analyzed at Vᵣ=0V on the 10 mM KCl—Ag/AgCl scale, then again at −223 mV on the same scale, and then returned to 0V and re-analyzed. The results (FIG. 11) duplicated those presented in FIG. 2D, demonstrating the reversibility of these measurements.

### Example 3

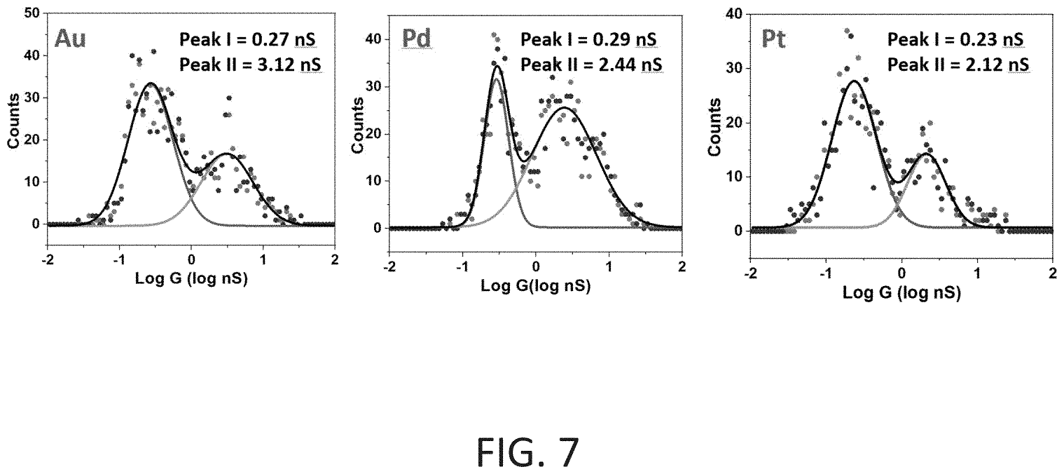

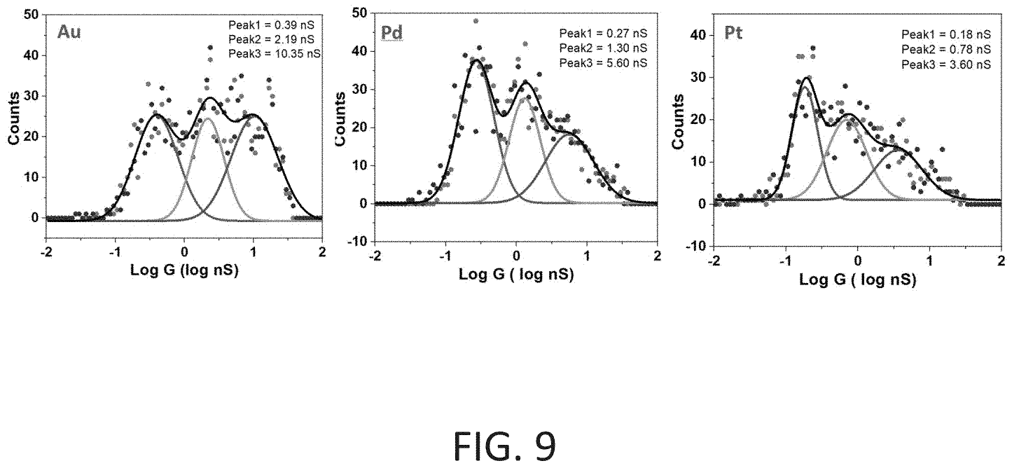

Resonances in other non-redox active proteins. Bivalent antibodies make excellent electrical contacts to electrodes functionalized with small epitopes, so measurements were repeated using electrodes coated with a thiolated dinitrophenol (DNP) molecules that captured anti-DNP IgE molecules. Another system of technological importance is a doubly-biotinylated Φ29 polymerase trapped between streptavidin-coated electrodes. The streptavidin is connected to the electrodes using thiolated biotin (as in the example above). In both of these larger systems, the gap size was set to 4.5 nm. The antibody conductance distribution consists of two peaks (FIG. 7). The lower conductance peak (peak I) arises from one specific and one non-specific contact and it is dominated by contact resistance. This peak is unaffected by the carrier potential (red and green points in FIG. 3A). Peak II arises from two specific contacts and has a much smaller contribution from contact resistance. This second peak depends strongly on potential. The peak of a Lorentzian fit to this potential dependence is again near 300 mV (Table 2). The polymerase distributions contain three peaks (FIG. 9) of which peaks II and III are sensitive to conformational changes in the protein. These two peaks are both affected by carrier potential, as shown in FIG. 3B. Once again, the conductance peaks at a potential near 300 mV vs NHE. Fitting parameters are given in Table 2.

TABLE 2
Parameters of the Lorentzian resonance in 3 proteins.
The peak width here is equal to 217 in equation 3.

| | Peak Energy | Peak Width | |
|---|---|---|---|
| Sample | (mV vs NHE) | (mV) | R² |
| SH-biotin/SA- | 287 ± 8 | 154 ± 28 | 0.991 |
| Electrochemical gating (Pk III) | | | |
| SH-biotin/SA- | 286 ± 5 | 90 ± 16 | 0.984 |
| Electrochemical gating (Pk II) | | | |
| SH-biotin/SA- | 301 ± 3 | 183 ± 43 | 0.994 |
| different metals (Pk III) | | | |
| SH-DNP/Ab- | 319 ± 10 | 72 ± 33 | 0.953 |
| different metals (Pk II) | | | |
| SH-biotin/SA/029- | 259 ± 6 | 183 ± 33 | 0.989 |
| different metals (Pk III) | | | |
| SH-biotin/SA/029- | 263 ± 13 | 189 ± 64 | 0.962 |
| different metals (Pk II) | | | |

Additional support for the various embodiments described herein can be found in the following tables.

TABLE 3
Work functions of the metals before and after plasma cleaning.
Work Functions of Various Metals (eV)

| | Palladium | Platinum | Gold |
|---|---|---|---|
| As Received | 4.57 | 4.62 | 4.85 |
| Plasma Cleaned | 5.02 | 5.06 | 5.32 |

TABLE 4
Conductance measurement of streptavidin with different materials as the electrodes.

| | Peak I | | | | | | Peak III | | |
|---|---|---|---|---|---|---|---|---|---|
| | | | Half | Peak II | | | | | Half |
| Materials | Position | Height | Width | Position | Height | Width | Position | Height | Width |
| (Tip-Sub) | (nS) | (Counts) | (Log G) | (nS) | (Counts) | (Log G) | (nS) | (Counts) | (Log G) |
| Au-Au | 0.41 ± 0.02 | 26.89 ± 1.63 | 0.42 ± 0.03 | 2.88 ± 0.11 | 32.89 ± 1.60 | 0.58 ± 0.04 | 13.45 ± 0.62 | 18.42 ± 2.30 | 0.24 ± 0.04 |
| Pd-Pd | 0.26 ± 0.01 | 52.75 ± 1.83 | 0.60 ± 0.04 | 1.50 ± 0.08 | 40.99 ± 2.93 | 0.41 ± 0.05 | 6.80 ± 0.63 | 31.76 ± 1.92 | 0.59 ± 0.08 |
| Pt-Pt | 0.17 ± 0.01 | 26.46 ± 0.96 | 0.56 ± 0.04 | 0.91 ± 0.07 | 16.04 ± 0.99 | 0.49 ± 0.11 | 3.63 ± 0.44 | 9.80 ± 1.51 | 0.38 ± 0.08 |
| Au-Pd | 0.30 ± 0.01 | 31.47 ± 0.83 | 0.50 ± 0.02 | 3.05 ± 0.07 | 24.59 ± 0.93 | 0.42 ± 0.02 | 16.26 ± 0.75 | 10.59 ± 0.94 | 0.38 ± 0.05 |
| Au-Pt | 0.40 ± 0.02 | 20.13 ± 0.92 | 0.62 ± 0.06 | 2.74 ± 0.55 | 14.01 ± 5.05 | 0.46 ± 0.18 | 8.13 ± 1.27 | 10.02 ± 5.52 | 0.46 ± 0.20 |
| Pt-Pd | 0.20 ± 0.01 | 27.82 ± 0.85 | 0.50 ± 0.02 | 1.03 ± 0.01 | 29.16 ± 1.24 | 0.22 ± 0.01 | 3.91 ± 0.17 | 12.95 ± 0.90 | 0.46 ± 0.04 |
| Pd-Au | 0.30 ± 0.01 | 22.01 ± 1.04 | 0.38 ± 0.02 | 2.96 ± 0.14 | 29.90 ± 0.92 | 0.58 ± 0.04 | 15.07 ± 0.70 | 17.68 ± 1.15 | 0.43 ± 0.04 |
| Pt-Au | 0.29 ± 0.01 | 26.67 ± 1.10 | 0.57 ± 0.03 | 2.65 ± 0.12 | 23.95 ± 1.42 | 0.43 ± 0.04 | 9.45 ± 1.41 | 10.16 ± 1.61 | 0.55 ± 0.07 |
| Pd-Pt | 0.20 ± 0.01 | 23.99 ± 0.81 | 0.53 ± 0.02 | 1.11 ± 0.03 | 16.38 ± 1.16 | 0.24 ± 0.03 | 3.70 ± 0.14 | 16.11 ± 0.90 | 0.43 ± 0.04 |

TABLE 5
Conductance measurement of phi29 with different materials as the electrodes.

| | Peak I | | | | | | Peak III | | |
|---|---|---|---|---|---|---|---|---|---|
| | | | Half | Peak II | | | | Height | |
| Materials | Position | Height | Width | Position | Height | Width | Position | (Counts) | Half |
| (Tip-Sub) | (nS) | (Counts) | (Log G) | (nS) | (Counts) | (Log G) | (nS) | (Log G) | Width |
| Au-Au | 0.39 ± 0.03 | 26.04 ± 1.30 | 0.62 ± 0.07 | 2.19 ± 0.15 | 25.16 ± 1.84 | 0.47 ± 0.06 | 10.35 ± 0.72 | 25.74 ± 1.56 | 0.68 ± 0.08 |
| Pd-Pd | 0.27 ± 0.01 | 37.35 ± 1.24 | 0.50 ± 0.02 | 1.30 ± 0.09 | 27.46 ± 1.52 | 0.44 ± 0.03 | 5.60 ± 0.52 | 18.04 ± 1.23 | 0.68 ± 0.06 |
| Pt-Pt | 0.18 ± 0.004 | 26.71 ± 1.26 | 0.36 ± 0.02 | 0.78 ± 0.05 | 18.95 ± 1.12 | 0.55 ± 0.07 | 3.60 ± 0.33 | 11.84 ± 0.99 | 0.67 ± 0 |
| Au-Pd | 0.26 ± 0.01 | 20.12 ± 0.77 | 0.74 ± 0.04 | 2.57 ± 0.30 | 10.43 ± 0.88 | 0.54 ± 0.12 | 11.22 ± 0.76 | 9.85 ± 0.94 | 0.43 ± 0.06 |
| Au-Pt | 0.24 ± 0.02 | 18.08 ± 0.75 | 0.73 ± 0.06 | 1.78 ± 0.16 | 11.27 ± 1.36 | 0.52 ± 0.09 | 7.94 ± 1.85 | 6.34 ± 1.12 | 0.81 ± 0.15 |
| Pt-Pd | 0.20 ± 0.01 | 16.57 ± 0.76 | 0.52 ± 0.05 | 0.86 ± 0.07 | 11.91 ± 1.30 | 0.40 ± 0.08 | 3.95 ± 0.32 | 15.02 ± 0.72 | 0.60 ± 0.06 |
| Pd-Au | 0.34 ± 0.01 | 23.68 ± 1.04 | 0.47 ± 0.03 | 2.4 ± 0.11 | 24.12 ± 0.91 | 0.55 ± 0.08 | 12.02 ± 0.33 | 22.62 ± 1.14 | 0.38 ± 0.03 |
| Pt-Au | 0.25 ± 0.01 | 42.33 ± 1.06 | 0.47 ± 0.02 | 1.67 ± 0.09 | 18.62 ± 1.09 | 0.51 ± 0.06 | 7.82 ± 0.92 | 9.27 ± 1.28 | 0.50 ± 0.12 |
| Pd-Pt | 0.21 ± 0.01 | 26.38 ± 1.01 | 0.44 ± 0.03 | 1.04 ± 0.08 | 13.88 ± 0.99 | 0.51 ± 0.10 | 4.46 ± 0.56 | 10.67 ± 1.44 | 0.59 ± 0.05 |

TABLE 6
Conductance measurement of anti-DNP antibody with different materials.

| | Peak I | | | Peak II | | |
|---|---|---|---|---|---|---|
| Materials | Position | Height | Half Width | Position | Height | Half Width |
| (Tip-Sub) | (nS) | (Counts) | (Log G) | (nS) | (Counts) | (Log G) |
| Au-Au | 0.27 ± 0.01 | 33.64 ± 1.18 | 0.63 ± 0.03 | 3.12 ± 0.21 | 17.06 ± 1.13 | 0.72 ± 0.08 |
| Pd-Pd | 0.29 ± 0.01 | 31.34 ± 1.29 | 0.33 ± 0.02 | 2.44 ± 0.1 | 25.35 ± 0.83 | 0.88 ± 0.04 |
| Pt-Pt | 0.23 ± 0.01 | 27.03 ± 0.88 | 0.59 ± 0.03 | 2.12 ± 0.1 | 13.52 ± 0.93 | 0.51 ± 0.05 |
| Au-Pd | 0.29 ± 0.01 | 35.49 ± 1.31 | 0.45 ± 0.02 | 4.16 ± 0.17 | 21.31 ± 1.16 | 0.60 ± 0.04 |
| Au-Pt | 0.20 ± 0.01 | 26.33 ± 0.92 | 0.65 ± 0.03 | 2.95 ± 0.16 | 15.79 ± 0.90 | 0.68 ± 0.05 |
| Pt-Pd | 0.26 ± 0.01 | 26.81 ± 0.91 | 0.63 ± 0.03 | 2.57 ± 0.13 | 15.08 ± 0.98 | 0.51 ± 0.04 |
| Pd-Au | 0.36 ± 0.01 | 37.69 ± 1.23 | 0.54 ± 0.02 | 4.97 ± 0.15 | 28.36 ± 1.19 | 0.59 ± 0.03 |
| Pt-Au | 0.28 ± 0.01 | 22.22 ± 0.99 | 0.51 ± 0.03 | 2.75 ± 0.12 | 19.35 ± 0.92 | 0.71 ± 0.05 |
| Pd-Pt | 0.27 ± 0.01 | 31.91 ± 1.07 | 0.67 ± 0.03 | 2.29 ± 0.19 | 12.45 ± 1.12 | 0.55 ± 0.04 |

TABLE 7
Conductance as a function of surface polarization measurement of streptavidin system (Pd-Pd).

| | Peak I | | | Peak II | | | Peak III | | |
|---|---|---|---|---|---|---|---|---|---|
| Polarized | | | Half | | | Half | | | Half |
| potential | Position | Height | Width | Position | Height | Width | Position | Height | Width |
| (mV) | (nS) | (Counts) | (Log G) | (nS) | (Counts) | (Log G) | (nS) | (Counts) | (Log G) |
| −223 mV | 0.28 ± 0.01 | 39.74 ± 1.15 | 0.45 ± 0.02 | 3.02 ± 0.32 | 17.82 ± 1.09 | 0.61 ± 0.09 | 13.49 ± 1.41 | 12.96 ± 2.01 | 0.41 ± 0.07 |
| −200 mV | 0.28 ± 0.01 | 35.00 ± 1.24 | 0.37 ± 0.02 | 3.31 ± 0.25 | 17.14 ± 0.97 | 0.80 ± 0.05 | 13.90 ± 1.24 | 7.96 ± 1.47 | 0.39 ± 0.02 |
| −158 mV | 0.31 ± 0.01 | 42.85 ± 1.10 | 0.42 ± 0.02 | 3.96 ± 0.25 | 21.28 ± 1.12 | 0.75 ± 0 | 16.50 ± 1.06 | 9.86 ± 1.32 | 0.40 ± 0.06 |
| −100 mV | 0.24 ± 0.01 | 28.78 ± 1.08 | 0.61 ± 0.04 | 1.99 ± 0.09 | 21.70 ± 1.23 | 0.51 ± 0.05 | 9.89 ± 0.40 | 16.60 ± 1.46 | 0.30 ± 0.04 |
| 0 mV | 0.23 ± 0.01 | 26.96 ± 1.12 | 0.65 ± 0.05 | 1.50 ± 0.08 | 25.48 ± 1.51 | 0.41 ± 0.05 | 6.03 ± 0.63 | 15.79 ± 1.36 | 0.47 ± 0.08 |
| +120 mV | 0.27 ± 0.01 | 26.71 ± 0.86 | 0.70 ± 0.01 | 1.29 ± 0.05 | 16.42 ± 1.53 | 0.24 ± 0.03 | 3.92 ± 0.21 | 15.73 ± 1.15 | 0.41 ± 0.05 |

TABLE 8
Reversibility measurement with polarization control.

| | Peak I | | | Peak II | | | Peak III | | |
|---|---|---|---|---|---|---|---|---|---|
| Polarized | | | Half | | | Half | | | Half |
| potential | Position | Height | Width | Position | Height | Width | Position | Height | Width |
| (mV) | (nS) | (Counts) | (Log G) | (nS) | (Counts) | (Log G) | (nS) | (Counts) | (Log G) |
| 1^{st} 0 mV | 0.26 ± 0.01 | 29.49 ± 0.93 | 0.50 ± 0.02 | 1.45 ± 0.04 | 24.74 ± 1.08 | 0.36 ± 0.03 | 6.11 ± 0.26 | 15.40 ± 1.03 | 0.39 ± 0.04 |
| 2^{nd}-223 mV | 0.29 ± 0.01 | 33.86 ± 1.23 | 0.52 ± 0.02 | 3.00 ± 0.12 | 24.00 ± 1.30 | 0.50 ± 0.04 | 13.72 ± 1.08 | 13.34 ± 1.46 | 0.43 ± 0.05 |
| 3^{rd} 0 mV | 0.26 ± 0.01 | 35.37 ± 1.16 | 0.44 ± 0.02 | 1.47 ± 0.04 | 30.18 ± 1.24 | 0.44 ± 0.03 | 6.61 ± 0.19 | 21.23 ± 1.39 | 0.29 ± 0.03 |

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A bioelectronic device comprising:
a first electrode and a second electrode separated by a gap; and
a protein attached to the first and second electrodes via a linker;
wherein the electrical surface potential of the first and second electrodes at zero bias is from about 250 mV to about 400 mV on the normal hydrogen electrode scale.

2. The device of claim 1, wherein conductance of the protein is maximized when the surface potential of the first and second electrodes at zero bias is from about 250 mV to about 400 mV.

3. The device of claim 1, wherein the surface potential of the first and second electrodes at zero bias is from about 250 mV to about 350 mV.

4. The device of claim 1, wherein the first and second electrodes are comprised of a metal or metals that impart a surface potential from about 250 mV to about 400 mV at zero bias.

5. The device of claim 1, wherein at least one of the first and second electrodes comprises a different metal as that of the other electrode.

6. The device of claim 1, wherein at least one of the first and second electrodes comprises gold or an alloy thereof.

7. The device of claim 1, wherein both the first and second electrodes comprise gold or an alloy thereof.

8. The device of claim 1, wherein the first electrode comprises gold or an alloy thereof and the second electrode comprises a different metal or an alloy thereof.

9. The device of claim 8, wherein the second electrode comprises palladium or an alloy thereof.

10. The device of claim 8, wherein the second electrode comprises platinum or an alloy thereof.

11. The device of claim 1, wherein the device comprises a reference electrode, and wherein the surface potential of the first and second electrodes is maintained at about 250 mV to about 400 mV due to a bias applied between the reference electrode and at least one of the first or second electrode.

12. The device of claim 11, wherein the reference electrode comprises a third electrode immersed in an electrolyte solution and in contact with the first and second electrodes.

13. The device of claim 1, wherein the gap has a width of about 1.0 nm to about 20.0 nm.

14. The device of claim 1, wherein the first and second electrodes are separated by a dielectric layer.

15. The device of claim 1, wherein the protein is a non-redox protein.

16. The device of claim 15, wherein the protein is selected from the group consisting of a polymerase, a nuclease, a proteasome, a glycopeptidase, a glycosidase, a kinase and an endonuclease.

17. The device of claim 1, wherein the linker is attached to an inactive region of the protein.

18. The device of claim 1, wherein the linker comprises a covalent chemical bond.

19. The device of claim 1, wherein the protein is biotinylated.

20. The device of claim 1, wherein the linker comprises thio-streptavidin.

21. The device of claim 1, wherein the protein and the first and second electrodes are biotinylated, and wherein the linker comprises a streptavidin molecule comprising at least two biotin binding sites.

22. A system for direct electrical measurement of protein activity, the system comprising:
(a) the bioelectronic device of claim 1;
(b) a means for introducing an analyte capable of interacting with the protein;
(c) a means for applying a voltage bias between the first and second electrodes that is 100 mV or less; and
(d) a means for monitoring fluctuations that occur as the chemical entity interacts with the protein.

23. An array comprising a plurality of the bioelectronic devices of claim 1.

24. The array of claim 23, wherein the array comprises:
(a) a means for introducing an analyte capable of interacting with the protein;
(b) a means for applying a voltage bias between the first and second electrodes that is 100 mV or less; and
(c) a means for monitoring fluctuations that occur as the chemical entity interacts with the protein.

25. A method for direct electrical measurement of protein activity, the method comprising
(a) introducing an analyte capable of interacting with the protein to the device of claim 1;
(b) applying a voltage bias between the first and second electrodes that is 100 mV or less; and
(c) observing fluctuations in current between the first and second electrodes that occur when the analyte interacts with the protein.

26. The method of claim 25, wherein the analyte is a biopolymer selected from the group consisting of a DNA molecule, an RNA molecule, a peptide, a polypeptide, or a glycan.
